(19) United States
(12) Patent Application Publication
(10) Pub. No.: US 2012/0045835 A1
(43) Pub. Date: Feb. 23, 2012

(54) PORTABLE DEVICE BASED ON IMMOBILIZED CELLS FOR THE DETECTION OF ANALYTES

(76) Inventors: Elisa Michelini, Bologna (IT); Aldo Roda, Bologna (IT); Luisa Stella Dolci, Bologna (IT); Luca Cevenini, Bologna (IT); Laura Mezzanotte, Bologna (IT)

(21) Appl. No.: 13/201,333

(22) PCT Filed: Feb. 11, 2010
(86) PCT No.: PCT/IB10/50625

§ 371 Date: Oct. 14, 2011

(30) Foreign Application Priority Data
Feb. 13, 2009 (IT) RM2009A000064

(51) Int. Cl. C12N 5/071; C12N 1/20; C12N 1/16; C12M 1/34

(52) U.S. Cl. 435/404; 435/287.1; 435/288.7; 435/253.6; 435/256.8

(57) ABSTRACT

The present invention relates to a portable device for the detection of analytes, in particular toxic substances, comprising at least a whole-cell biosensor with cells immobilized onto a transparent and inert matrix that allows the maintenance of cell vitality. The matrix comprises a mixture of collagen and/or its enzyme degradation derivatives, a proteoglycan and a mixture of vinyl polymer and an optionally modified polysiloxane, and an orthosilicate. The biosensor can be a genetically modified cell that expresses luciferase as reporter gene.

The present invention relates to environmental, food and clinical analysis fields and provides a portable device for the multiplexed analysis of toxic substances. In particular, the present invention provides a portable device that includes a panel of cells immobilized onto a biocompatible matrix that preserves their vitality. This matrix allows the immobilization of different genetically engineered cells.

### BACKGROUND OF THE INVENTION

Biosensors are analytical devices or biosensing systems in which a biological component specifically recognizes and binds the target analyte. This biological component in turn is coupled to a physical transducer, capable of producing a measurable analytical signal. Among biosensors, genetically engineered cell-based sensing systems can be obtained by providing the cell with a reporter protein, whose expression is controlled by regulatory proteins and promoter (O/P) sequences. The regulatory protein is able to recognize the presence of the analyte and to consequently activate the expression of the reporter protein. The reporter protein can then be readily measured and directly related to the analyte bioavailability in the sample.

Cell-based biosensors rely on cell capacity of responding to the presence of chemicals by modifying their metabolism as a consequence of the interaction. One of the main advantages of cell-based biosensors that employ reporter genes is reduction of detection limits due to signal transduction cascades. Besides, the use of complex recognition systems such as cells allowed expanding the applicability field of biosensors because, unlike conventional analytical techniques, permits to evaluate bioavailability and biological effects of target analytes.

Cell-based biosensors can be distinguished in two classes: those employed for general toxicity indicators and those used to detect and quantify specific target analytes.

In the first case, variations in cell metabolism and cell respiration have been exploited to evaluate toxic effects of environmental samples or target analytes; in some works marine bacteria able to spontaneously emit bioluminescence have been used to evaluate, through luminescence reduction, cell death due to sample toxicity.

In the second case, cells have been employed for detecting a specific analyte or a class of analytes. For this purpose, cell-based biosensors have been obtained by recombinant DNA techniques. Besides, by employing whole cells or tissues it is possible to study “group effects” such as toxicity, mutagenicity and pharmacological activity.

As fungi, bacteria present, the advantage of being ubiquitous and they are able to grow also in non-optimal conditions and metabolize a wide range of chemical compounds, both in aerobic and anaerobic conditions. Several chemicals are metabolized and transformed by bacteria in products easily to measure such as ammonia, carbon dioxide and acids.

The maintenance of bacterial biosensors is usually cost-effective since bacterial culture reagents and necessary equipment are inexpensive. The relative simplicity that allows to genetically engineer bacteria and the availability of several reporter genes led to the development of a number of recombinant cell-based biosensors that respond to a specific analyte or that can be used as general toxicity or genotoxicity indicators.

To act as “microbial bioreporters”, a bacterial cell should contain two genetic elements: a sensitive element and a reporter gene. The sensitive element detects the presence of a target molecule and activates the reporter gene which expression produces a measurable signal. The reporter element is usually a gene that encodes for a protein which presence or activity is easily measurable.

The sensitive element is different in each bacterial biosensor and its choice accounts for the uniqueness and specificity of the final construct. In most cases, the sensitive element is a promoter specific for a gene, a receptor or a group of genes which are normally activated in response of a specific environmental change.

In intact biological systems, target gene activation would lead to the expression of proteins required for cell survival or adaptation to novel environmental conditions.

Otherwise, in a recombinant system, activation of target genes leads to the synthesis of one or more reporter proteins, which expression is easy to measure. To obtain specificity for a target analyte, a regulatory mechanism should be introduced in the cell in a way that reporter protein expression is driven by activation of the promoter sequence. This goal can be easily achieved if the regulatory element (e.g., receptor) is already present in the host cell. The genetic construct promoter/reporter gene can be introduced in a host cell by two different mechanisms: integrated or in episomial form.

Usually, plasmids are maintained in episomial form with antibiotic selection for bacterial biosensors but, in some situations, such as on field environmental monitoring, this selection is not suitable neither attractive due to potential release in the environment of antibiotic resistance markers. Plasmid integration into the host bacteria chromosome allows a stable expression of heterologous DNA, nevertheless a site-specific integration is difficult to obtain and thus the risk of interruption of the host chromosome cannot be ruled out.

The use of recombinant DNA techniques allows developing bacterial biosensors with a plethora of reporter genes encoding for proteins or enzymes detectable with several principles. In particular, genes encoding for proteins which expression is measured by bio-chemiluminescence (bacterial or firefly luciferase) or fluorescence are gaining more interest.

Bio-chemiluminescence detection guarantees high sensitivity and specificity, while fluorescence measurements are very easy to perform since they do not need any reagent addition and instrumentations are reasonably simple and commercially available.

Monitoring of contaminated areas is a huge issue that involves local and international authorities. An example is the emerging problem related to electronic discards (e-waste), such as computers, televisions, mobiles, copiers, and fax machines. Their presence in the environment can release toxic components, such as heavy metals, dioxins, and other endocrine disruptors such as polychlorinated biphenyls (PCB) and aromatic hydrocarbons.

E-waste is routinely exported by developed countries to developing ones, and frequently in violation of the international law. The processing of e-waste in developing countries poses serious health and pollution problems. The real-time identification of contaminated sites, and the (semi-) quantitative detection of specific analytes is surely of primary importance for the remediation of contaminated sites and avoid risk to the environment and human health. Biosensors that emit a bioluminescent signal in the presence of toxic compounds have been proposed and their analytical performance is rapidly improving thank to their continuous optimization.

Michelini E., Leskinen P., Virta M., Karp M., Roda A.; Biosensors & Bioelectronics 2005, 20, 2261-2267 describe a bioluminescent assay for the detection of compounds with androgenic activity. The biosensor is a recombinant S. cerevisiae. This biosensor is employed in laboratories with cell culture facilities. See also Leskinen P., Michelini E., Picard D., Karp M., Virta M.; Chemosphere 2005, 61, 259-266.

Mirasoli M., Feliciano J., Michelini E., Daunert S., Roda A.; Anal. Chem. 2002, 74, 5948-5953 describe a bacterial biosensor with an internal signal correction obtained by introducing an additional reporter gene that allows to correct the analytical signal according to aspecific effects due to toxicity of the matrix or changes in experimental conditions.

One of the main problems of biosensors is cells' vitality. This, together with the need for special equipment and cell culture facilities, complicates their use for on field applications. Therefore samples to be analyzed must be sent to specially equipped laboratories, even at big distances, and the analysis must be performed by skilled personnel.

Thus, there is an urgent need for a portable, cost-effective and easy-to-use system to detect toxic substances. This system should maintain cells' vitality and, as a consequence, the accuracy of the measurements directly on the field.

Patent application US 2005/157304 discloses a portable device for the measurement of a variety of chemical and biological substances. The device is based on optical detection and does not employ biosensors.

D'Souza S F (Microbial biosensors. Biosens Bioelectron. 2001 August; 16(6):337-53) discloses the entrapment of viable cells in polymeric matrices for the manufacture of stable bioluminescent cell biosensors. For example, the microbial cells are entrapped in a matrix selected from polyvinyl alcohol, albumin-PEG-hydrogels, alginate, carrageenan, agarose, chitosan, or the cells adhere on a cellulose surface. A further improvement in stability is achieved by reinforcing an alginate matrix with polyacrylamide.

Simpson M L, Sayler G S, Ripp S, Nivens D E, Applegate B M, Paulus M J, Jellison G E (1998) Bioluminescent bioreporters integrated circuits form novel whole-cell biosensors. Trends Biotech 16:332-338 describe bioluminescent microbial biosensors, in which cells are encapsulated in a polymeric matrix to increase the biosensor stability. The polymeric matrix is selected from agar/agarose, carrageenan, alginate, polyurethane-polycarbonyl sulfonate, polyacrylamide, polyvinyl alcohol and a sol-gel composition, which is obtained from the solidification of silanes, siloxanes and orthosilicates.

Patent TW 239392 discloses a portable biosensing system combined with specific signal processing to detect water toxicity or nutritive properties. The system is based on the inhibition of microbial growth due to sample toxicity. Inhibition is revealed by current through biochemical or electro-chemical reaction on the electrode.

Patent application US 2008/032326 discloses a water quality analyzer based on an electro-osmosis cell and a plurality of photosynthetic organisms. The presence of toxic substances reduces their photosynthetic activity, which is measured as index of the presence of toxicity.

To date, portable devices based on immobilized bioluminescent whole-cell biosensors for on-field analysis have not yet been implemented in the market. The main advantage of bioluminescence is its higher sensitivity due to absence of aspecific signal (such as in fluorescence detection). Besides, bioluminescence does not require external light source, thus greatly simplifying the instrumentation for signal acquisition. The high quantum efficiency of luciferase from firefly with respect to luciferase isolated from other organisms, such as that one isolated from bacteria as in application WO2007083137, allows to achieve limits of detection of 10⁻¹⁹ mol luciferase.

Application WO2007083137 discloses a device composed of biosensors able to detect a specific analyte on the basis of the emission of volatile substances, an immobilization procedure is envisaged based on the use of a matrix of agar, agarose and alginate, all components commonly used for the immobilization of bacterial cells.

Application WO2008152124 discloses an engineered yeast cell used as biosensor.

Patent US 2003/162164A1 discloses a testing system in which cells are fixed onto a multiwell support by means of a suspension medium preferably containing: a polysaccharidic gelling agent, such as carrageenan or alginate, a suspending agent selected from agarose, cellulose, polyvinyl-alcohol, collagen, silicone, etc and a transferable matrix, e.g., cellulose.

The issue related to the use of a portable biosensor for the detection of a multiplicity of toxic substances (or substances endowed with specific biological activity) based on immobilized cells that maintain cell vitality has not been solved yet.

Besides, another problem consists in potential contamination due to the release in the environment of viable genetically engineered cells.

Despite continuous efforts for improving biosensors, until now whole-cell biosensors have not been made available for on-field analysis outside laboratories. The main difficulty consists in finding a suitable immobilization procedure that keeps vitality of different cell types and cells engineered with different constructs. An ideal matrix should maintain cell viability even in uncontrolled experimental conditions such as those found in external environmental. The only portable devices reported in literature are BBIC (Bioluminescent Bioreporter Integrated Circuits), but they use bacterial liquid cultures (Nivens D E, McKnight T E, Moser S A, Osbourn S J, Simpson M L, Sayler G S. Bioluminescent bioreporter integrated circuits: potentially small, rugged and inexpensive whole-cell biosensors for remote environmental monitoring. J. Appl. Microbiol. 2004; 96(1):33-46).

In the paper of Nivens et al., bacterial suspension (Pseudomonas fluorescens with lux cassette CDBAE) is simply added to the electric circuit before the luminescence measurement, without solving immobilization issues and confinement of genetically engineered cells.

### SUMMARY OF THE INVENTION

A new biocompatible matrix has now been found capable of solving whole-cell immobilization issues. The matrix keeps cell vitality and allows their incorporation into a portable device for the simultaneous multiparametric detection of a number of analytes.

This device is able to perform High Content Screening (HCS) analysis, allowing to obtain several information, such as the presence of different analytes (e.g., heavy metals, xenobiotics, endocrine disruptors) or information about biological or toxic activity and other information, from a single sample, e.g., environmental, food or clinical, by using, instead of conventional chemical-physical techniques, bioluminescent cells engineered to specifically respond to the presence of analytes of organic or inorganic nature.

The use of the device of the present invention, enables on-field monitoring of toxic compounds or substances with specific biological activity in environmental, food, or biological samples in general, without the necessity of expensive instrumentation or the need to transfer samples to equipped laboratories.

The present invention finds interesting applications in real-time on-field environmental analysis, allowing to rapidly obtaining quantitative data, which can be considered immediate if compared with laboratory timings.

Another interesting application of the device object of the invention is its use in developing countries and in those situations were specially-equipped laboratories are not available.

Therefore, it is an object of the present invention a portable device for the detection of toxic substances or compounds with biological activity comprising at least a biosensor, characterized in that the cells are immobilized on a matrix, as disclosed thereafter.

Genetically engineered bacterial or yeast cells are immobilized in the device by means of a new biocompatible matrix comprising a combination of synthetic and natural polymers functionalized with specific crosslinking agents. This formulation provides a transparent, inert three-dimensional structure able to encapsulate cells and maintain their vitality.

The transparent and inert matrix keeping cell vitality, according to the present invention, comprises:
a) a natural polymeric component, called natural component, which is a mixture of collagen and/or its derivates of enzymatic degradation, a proteoglycan, and optionally a vegetal mucilage and
b) a synthetic polymeric component, called synthetic component, which is a mixture of a vinyl polymer and a polysiloxane, optionally modified and/or optionally crosslinked with an orthosilicate.

As natural polymers, forming the biocompatible part which keeps whole-cell biosensor vitality while immobilizing it, the matrix, according to the present invention includes a mixture of collagen and/or its derivatives of enzymatic degradation and a proteoglycan.

As synthetic polymers, forming the fixing part of the biosensor to the device, ensuring both transparency, which is essential for transmission of light signal to the detector, and that cells remain attached and do not disperse into the environment, the matrix object of the present invention includes a vinyl polymer and a polysiloxane, optionally modified and/or optionally crosslinked with an orthosilicate.

The device object of the present invention presents a cartridge containing immobilized cells which is completely isolated from external environment and, after use, can be removed from the device and disposed with conventional procedures (e.g., it is enough to autoclave or burn the cartridge to obtain cell death). In addition, the immobilization matrix contains non biodegradable vitrifying components (polysilanes) which avoid unintentional or voluntary release of microorganisms in the environment.

It is another object of the present invention an immobilization matrix of a biosensor as specified above and in the following detailed description.

These and other objects of the present invention will be now explained in detail in the following description also with examples and drawings.

FIG. 1.A. is a schematic view showing a simplified form of the device according to the present invention. FIG. 1.B. shows a lateral view of the device with immobilized cells.

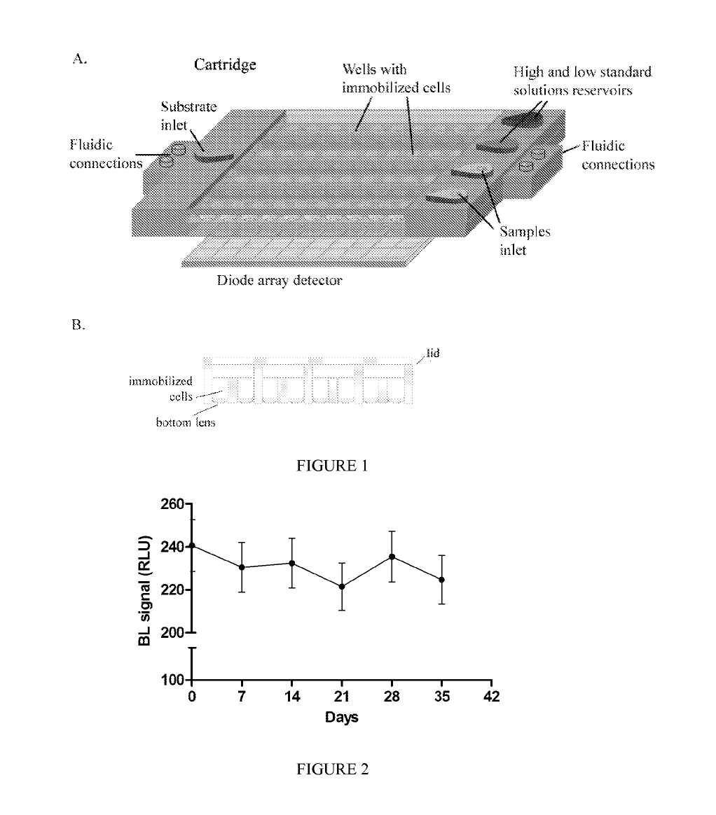

FIG. 2 is a graph showing light signal emitted from bioluminescent E. coli cells immobilized with the matrix object of the present invention.

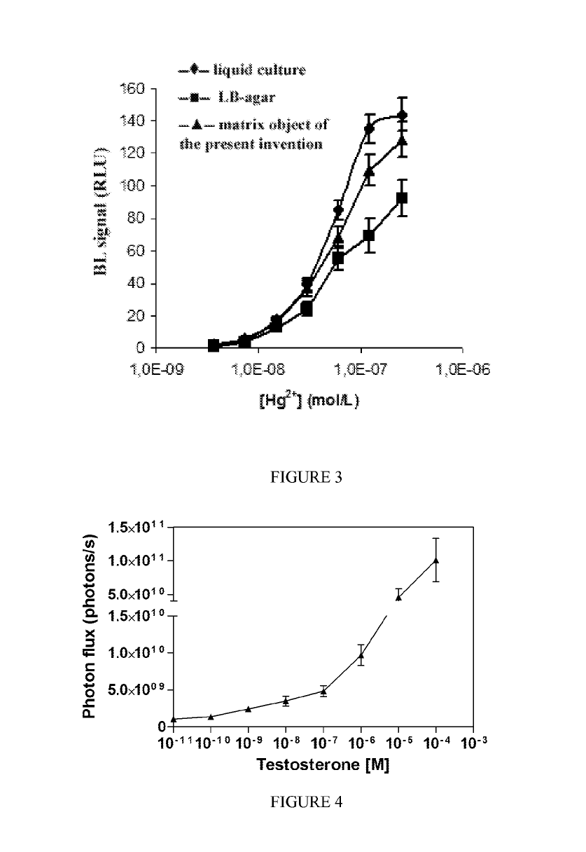

FIG. 3 is a graph showing Hg²⁺ concentrations detected with bacteria in liquid culture (—♦—), with bacteria immobilized with the matrix object of the present invention (—568 —) and with bacteria immobilized in conventional matrix (LB/agar —▪—).

FIG. 4 shows a calibration curve for testosterone obtained with the yeast strain responding to androgens immobilized in the matrix object of the invention.

### DETAILED DESCRIPTION OF THE INVENTION

The immobilization matrix according to the present invention is composed by a mixture of natural and synthetic polymers in varying proportions depending on the cell type.

As natural polymer, the present invention provides a combination of collagen and/or its derivatives obtained by its enzymatic degradation and proteoglycan, or a mixture of proteoglycans. Collagen can be of different nature and methods for its production and its various derivatives are well known and do not need further description. Purity and source of collagen (e.g., bovine, horse, pig, shark) must be compatible with the biosensor vitality and these requirements are well known to well-experienced in the field.

Analogously, collagen products derived by enzymatic digestion are well known and there are no particular limitations in their use in the present invention.

The same considerations apply for proteoglycan, which can be of different nature and purity, as long as they are compatible with the biosensor vitality.

As synthetic polymer, the present invention provides a combination of a synthetic vinyl polymer with an optionally modified polysiloxane.

The synthetic polymer combination must ensure both structural rigidity and biosensor confinement and sufficient transparency for optical signal transmission to the detector. On this base, a person of ordinary skill in the art can choose suitable polymers. Preferred example of vinyl polymer is polyvinylpyrrolidone (PVP). Proteoglycan is present at variable proportions in the range 10-25% w/w respect to collagen and/or its enzymatic degradation product.

In a first embodiment of the invention, the organic polymer comprises, or it preferably consists of, a mixture of collagen and/or collagen degradation products and a proteoglycan.

As an example, collagen degradation product is atelocollagen and proteoglycan is decorin.

A form of atelocollagen suitable for the aims of the present invention is that obtained by digestion of collagen type I with pepsin. This compound, thanks to its peculiar properties (liquid at 4° C. but gels after at 37° C. and has low toxicity) has been previously used as carrier substance (Ochiya T, Takahama Y, Nagahara S, et al. New delivery system for plasmid DNA in vivo using atelocollagen as a carrier material: the Minipellet. Nat. Med, 5: 707-10, 1999; “Biodegradable microcapsules having walls composed of crosslinked atelocollagen and polyholoside” U.S. Pat. No. 5,395,620) and in the composition of contact lenses (U.S. Pat. No. 4,687,518—Method for manufacturing pyrogen-free collagen gels useful as contact lenses), but it has never been used for immobilization of living cells.

Different cell types are mixed separately with the matrix in the liquid state to obtain homogeneous cell suspensions which are then dropped, also automatically, inside the wells of the cartridge and left at room temperature for a time sufficient for complete polymerization (from 30 min to 1 hr)

Conveniently, the immobilization procedure is automated to guarantee the best reproducibility of the device.

Generically, cells are grown in a liquid culture medium which is specific for each strain until to an optical density at 600 nm of 0.6-0.8. Then, a buffered solution at pH 6.5-7.0 is added to each cell suspension containing nutrients and is mixed 1:2-1:4 with the immobilization matrix.

Obviously, a skilled person is able to choose the more appropriate buffered solution according to the cell system of the biosensor.

In one embodiment of the invention, a solution containing 0.05 to 5% atelocollagen in PBS (NaCl 137 mM, KCl 2.7 mM, NaH₂PO₄ 1.4 mM, Na₂HPO₄ 4.3 mM) at pH 7.4 with a proteoglycan (for example decorin) concentration ranging from 10 to 25% w/w with respect to atelocollagen.

Besides, synthetic polymers, are added to the immobilization mixture, which is kept at 4° C. before cells addition to the device. The polymers, such as polyvinylpyrrolidone (PVP), and polysiloxanes, optionally modified and/or crosslinked with an orthosilicate, are used at different concentrations, typically from 0.05 to 15%, to create a matrix suitable to encapsulate cells and maintain their vitality, meanwhile assuring transparency, which is a crucial factor for the detection of luminescent signals.

As modified polysiloxane is intended a polysiloxane with alkyl, acrylate, alcohol groups. Polysiloxanes are polymers with a main backbone Si—O—Si of 30-60 Si atoms length and lateral chains from C₁ to C₁₂, for each polymer an average of 15-30 lateral chains is calculated.

An orthosilicate is a suitable crosslinking agent.

Preferred polysiloxane is dimethylsiloxane. Preferred crosslinking agent is tetraethyl-orthosilicate.

In another embodiment of the matrix object of the present invention, it is possible to use a vegetal mucilage to increase adhesiveness of the matrix to the substrate (well or any suitable compartment). A small percentage is enough. Examples of vegetal mucilages are those commercially available from mauve and aloe.

Preparation of the Matrix May Occur in an Automated Way Starting from Single Components Held in separate containers. Mixing of the matrix with different cell cultures can be automated as the addition of suspensions matrix/cells to single wells of the device.

Polymerization process can be started inside a robot and completed at +4-8° C. Reservoirs with cell cultures and polymer components are supplied manually.

According to the scopes of the present invention, a “biosensor” is an analytical device with a biological recognition element (e.g., receptor, antibody, cell, tissue) integrated with a transducer that converts biological response in a detectable event. A whole-cell biosensor is composed of a cellular system able to take chemicals and modify its own metabolism as a consequence of this interaction, leading to a detectable event.

A detectable event is a signal which can be detected and if possible quantified. An example of detectable event is bioluminescent emission. Also other detection types can be used in the present device, such as fluorescent, colorimetric, electrochemical, electro-chemiluminescent, acoustic.

In the device according to the present invention, the biosensor is preferably a multiplicity of biosensors that respond to a variety of chemicals and toxic substances. Advantageously, this multiplicity of biosensors allows the multiparametric and simultaneous determination of different analytes, in particular toxic substances.

In the preferred form of the device according to the present invention, the biosensor is a bacterial cell or yeast cell, but also other cell types can be used in the present device (e.g., human and mammalian cell lines, vegetal cell lines, algae, fungi).

In the preferred form, this biosensor is bioluminescent.

The biosensor can be spontaneously bioluminescent or is a cell genetically engineered with a reporter gene expressing a luciferase in the presence of at least one of the analytes, in particular toxic substances.

A luciferase is any bioluminescent protein (for example luciferase from P. pyralis, luciferase from Renilla reniformis, luciferase from Gaussia Princeps), i.e., able to catalyze a reaction that leads to light emission in the presence of a substrate, generically named luciferin (e.g., D-luciferin, Cypridina luciferin, coelenterazine).

Analytes that can be detected with the device object of the present invention are dependent upon the natural sensitivity of the biosensor to these substances, both in a specific way and in a general way (in this case to obtain a measure of general toxicity), or upon the type of cell genetically engineering. As a preferred example, toxic substances detected with the device of the present invention are: heavy metals, endocrine disruptors, xenobiotics, and genotoxic compounds.

In a preferred form, this biosensor s a cell genetically engineered with a reporter gene ex pressing a luciferase in the presence of at least one of these toxic compounds.

In a second preferred form, this biosensor is a multiplicity of biosensors for a plurality of toxic compounds. In such a way, the device object of the present invention allows the simultaneous and multiparametric detection of different substances.

The device object of the present invention can use different known biosensors, such as those divided into two classes: as general toxicity indicators and for the measure of specific analytes.

Ina preferred form, the device object of the present invention uses a multiplicity of biosensors specific for different analytes to provide a simultaneous multiparametric analysis.

In this embodiment, both natural biosensors, i.e. cells that naturally respond to specific analytes, and recombinant biosensors, i.e. obtained by genetic engineering, can be employed.

Preferably, the biosensor is a bacterial cell, thanks to the advantages provided by the use of bacteria. As an alternative, the biosensor is a yeast or mammalian cell. In particular, yeast or mammalian cells are used when it is necessary to express human or mammalian receptors that wound not be correctly expressed or folded in a prokaryotic cell (e.g., human receptors of steroid hormones).

The bacterial biosensor will be selected according to the target analyte and the determination of two linked genetic elements, i.e. the sensing element and the reporter one, is well known in the art; as well as techniques for the obtainment of the recombinant strain, some of them are also commercially available.

A preferred form of the biosensor, in particular a genetically engineered cell, uses the reporter gene technology, based on the activation of genes which expression is easily detected (e.g., with optical detection) and is regulated by specific events of transcriptional control. This technology allows obtaining cells modified to respond to specific analytes (or classes of analytes) with a luminescent signal proportional to bioavailable fraction of a specific analyte in contact with the cells.

The present invention allows to use a variety of biosensors, for example non pathogen bacterial cells (e.g., E. coli, B. subtilis, Ps. fluorescens, R. eutrophus) genetically engineered to respond to the presence of analytes or toxic events (e.g., oxidative stress, organic toxins, genotoxins, metals like cobalt, copper, mercury, nickel, zinc, organic compounds like naphthalene, 4-chlorobenzoate, benzene, toluene, ethylbenzene, organic peroxides, trichloroethylene, PCBs, salicylates) and yeast cells responding to compounds with pseudo-hormonal activity and xenobiotics. Following are some preferred embodiments of the invention.

Cells Genetically Engineered with a Reporter Gene Encoding for Luciferase

As stated, a preferred embodiment of the invention is based on the use of biosensors that show bioluminescence.

Besides the possibility of using cells that naturally show bioluminescence in the presence of toxic compounds, the present invention employs in a preferred way cells genetically engineered with a reporter plasmid encoding for a bioluminescent protein.

In a preferred form of the present invention, the luminescent protein encoded by the reporter gene is a luciferase.

Examples of luciferases that can be used with the present invention are: luciferase from firefly (e.g., P. pyralis, L. italica, described in US 2007/0190587), bacterial luciferase, luciferases from Renilla reniformis and Gaussia princeps, luciferases cloned from other organisms or obtained by mutagenesis. A preferred example of luciferase from Photinus pyralis is described in Branchini B., Southworth T., Khattak N., Michelini E., Roda A.; Analytical Biochemistry 2005, 345, 140-148. The expression of the reporter protein is regulated by a sequence which differs according to analyte specificity.

Other examples of luciferase that can be used for the present invention are described in Michelini E., Guardiagli M., Magliulo M., Mirasoli M., Roda A., Simoni P., Baraldini M.; Bioluminescent Biosensors Based on Genetically Engineered Living Cells in Environmental and Food Analysis. Anal. Lett., Vol. 39, Num. 8, 2006, 1503-1515.

A multiple system of luciferases can also be used as described in Michelini E., Cevenini L, Mezzanotte L., Ablamsky D., Southworth T., Branchini B, Roda A.; Photochem. Photobiol. Sci. 2008, 7, 212 e in Michelini E., Cevenini L., Mezzanotte L., Ablamsky D., Southworth T., Branchini B., Roda A.; Anal. Chem. 2008, 80(1), 260-267. By using more luciferases in the same cell it is possible to increase the throughput of the device, obtaining more information from the same cell. In this configuration of the device an additional filter system is required on the photodiodes grid to separate signals emitted from different luciferases. Alternatively an ultrasensitive color charge-coupled-device (CCD) camera can be used. This configuration allows to use another technique alternative to reporter gene technology: Bioluminescence Resonance Energy transfer (BRET), described in Michelini E, Mirasoli M, Karp M, Virta M, Roda A. Development of a bioluminescence resonance energy-transfer assay for estrogen-like compound in vivo monitoring. Anal Chem. 2004 Dec. 1; 76(23):7069-76.

### Bioluminescent Bacteria Specific for Heavy Metals:

Biosensors immobilized in the device object of the present invention are based on bacterial cells genetically engineered to express a regulatory protein as molecular recognition element specific for each metal to be analyzed (Hg²⁺, Cd²⁺, Zn²⁺, AsO₂⁺, Pb²⁺, Ni²⁺, Co²⁺, Sb³⁺⁽⁵⁺⁾, Cr³⁺⁽⁶⁺⁾, Cu²⁺, Sn²⁺⁽⁴⁺⁾ and others).

In a particular embodiment of the invention, different recombinant bacterial biosensors, each one specific for one or two metals, have been used.

Each of them contains a plasmid with the following elements:
a gene that provides resistance to a specific antibiotic, allowing selection and maintenance of cells harboring the plasmid in selective medium containing the antibiotic;
a reporter gene (luc) encoding for a luciferase;
a gene encoding for a regulatory protein, which is the specific molecular recognition element: once inside the cell, metal binds and activates the regulatory protein;
a specific gene sequence that, as a consequence of the interaction with the regulatory protein, activates transcription of the reporter gene. This sequence, together with regulatory protein, is the one that confers specificity to the biosensor.

Following the entrance of the target metal into the cell and its interaction with the regulatory protein, transcription and translation of the reporter gene start. Reporter protein, in the presence of the substrate luciferin, catalyzes a bioluminescent reaction, whose intensity is proportional to the bioavailable concentration of heavy metal present in the sample.

Since whole-cell biosensors are able to detect only the fraction of metal that enters into the cell, they provide information about the bioavailable fraction of metal in the sample. This analytical information, difficult to obtain with other analytical methods, is of particular interest to provide information about comprehensive ecotoxicological risk assessment of samples from areas under study. It is in fact well known that toxicity of a metal is not directly correlated to its total concentration, but it is more related to its bioavailable fraction, which is able to interact with a living organism.

Some examples of gene sequences that were introduced in bacterial cells (E. coli) to obtain specific biosensors for one or two heavy metals, as previously described in scientific literature, are reported in the following lines:
ars operon (containing ars promoter and ArsR repressor protein) isolated from Staphylococcus aureus (Regulation and expression of the arsenic resistance operon from Staphylococcus aureus plasmid pI258. Ji G, Silver S. J. Bacteriol. 1992 June; 174(11):3684-94; Recombinant luminescent bacteria for measuring bioavailable arsenite and antimonite. Tauriainen S, Karp M, Chang W, Virta M. Appl Environ Microbiol. 1997 November; 63(11):4456-61)—specificity for arsenite;
coa operon (containing the operator-promoter CoaT sequence and regulatory gene CoaR) isolated from Synechocystis sp PCC 6803 (Peca L, Kos P B, Mate Z, Farsang A, Vass I. specificity for cobalt and zinc.
Construction of bioluminescent cyanobacterial reporter strains for detection of nickel, cobalt and zinc. FEMS Microbiol Lett. 2008 December; 289(2):258-64.)—specificity for Co²⁺ and Zn²⁺;
nrs operon (containing the operator-promoter nrsB sequence and regulatory gene nrsR) (FEMS Microbiol Lett. 2008 December; 289(2):258-64. Construction of bioluminescent cyanobacterial reporter strains for detection of nickel, cobalt and zinc. Peca L, Kós PB, Máté Z, Farsang A, Vass I)— specificity for nickel;
cad operon (containing the operator-promoter Cad and regulatory gene CadR) (Biosensors and Bioelectronics. 13(9), 1998, 931-938. Luminescent bacterial sensor for cadmium and lead. Tauriainen S, Karp M., Changa W, Virta M)—specificity for cadmium and lead;
mer operon (containing the operator-promoter mer and regulatory gene merR) (Tauriainen S, Virta M, Chang W, Karp M. Measurement of Firefly Luciferase Reporter Gene Activity from Cells and Lysates Using Escherichia coli Arsenite and Mercury Sensors Anal Biochem. 1999 Aug. 1; 272(2):191-8.)—specificity for mercury;
promoter P_{pcoE} inducible with copper ions (Rouch D A, Parkhill J,. Brown N. L. Induction of bacterial mercury- and copper-responsive promoters: Functional differences between inducible systems and implications for their use in gene-fusions for in vivo metal biosensors. Journal of Industrial Microbiology and Biotechnology, 1995, 3-4)-specificity for copper.

Promoter sequences specific for each metal were introduced in suitable plasmids (known and some of them commercially available) upstream to the cDNA encoding for the bioluminescent reporter protein (e.g., luciferase from P. pyralis) to obtain an expression inducible by the analyte.

### Bioluminescent Yeast Cells Specific for Endocrine Disruptors:

The device relies on the immobilization of recombinant yeast cells, obtained by standard and widely described recombinant DNA techniques that respond to a variety of endocrine disruptors, xenobiotics, and genotoxic compounds.

For example, the device employs S. cerevisiae cells specific for compounds with estrogenic/androgenic activity: for this aim an integrative vector with a regulatory element containing two or more tandem copies of the Androgen or Estrogen Responsive Element (ARE/ERE) upstream of the gene encoding for the bioluminescent reporter protein (e.g., luciferase from P. pyralis) and a vector encoding for human androgen or estrogen receptor were introduced in S. cerevisiae cells (A new highly specific and robust yeast androgen bioassay for the detection of agonists and antagonists Toine F. H. Boyce, Richard J. R. Helsdingen, Astrid R. M. Hamers, Majorie B. M. van Duursen, Michel W. F. Nielen, and Ron L. A. P. Hoogenboom). In this work, GFP is used as fluorescent reporter gene that also finds application in the present invention.

To obtain the best expression of luciferase in yeast cells a luciferase without the peroxisomal targeting sequence Ser-Lys-Leu (SKL) is used. Many plasmids that contain this truncated for of luciferase are commercially available.

Other examples of cells that can be immobilized in the present device include:
S. cerevisiae cells specific for toxic compounds like dioxins: in these cells an expression plasmid encoding for the cellular receptor AhR (aryl hydrocarbon receptor) and ARNT (aryl hydrocarbon receptor nuclear translocator) were introduced as molecular recognition elements biospecific for the whole class of dioxin-like molecules.

In addition, an integrative vector (pRSXRELuc) containing two or more tandem copies of the regulatory sequence Dioxin Responsive Element (DRE) upstream of the gene encoding for luciferase has been inserted (Leskinen P, Hilsherova K, Sidlova T, Kiviranta H, Pessala P, Salo S, Verta M, Virta M Detecting AhR ligands in sediments using bioluminescent reporter yeast. Biosens. Bioelectron. 2008 Jul. 15; 23(12):1850-5).

Also spontaneously bioluminescent bacterial cells which are commercially available or described in scientific literature are used as reference strains for vitality control (Bioluminescent yeast assays for detecting estrogenic and androgenic activity in different matrices. Leskinen P, Michelini E, Picard D, Karp M, Virta M. Chemosphere. 2005 October; 61(2):259-66. A sensitive recombinant cell-based bioluminescent assay for detection of androgen-like compounds. Michelini E, Cevenini L, Mezzanotte L., Leskinen P, Virta M., Karp M, Roda A. Nature Protocols 2008, 3(12):1895-1902).

These cells are obtained by introduction into the genome of a vector for constitutive expression of luciferase. In normal conditions a stable level of luciferase is expressed independently from the presence of analytes. Only in the presence if interferents able to alter cell functionality and/or vitality or with cytotoxic effects a variation (increase or decrease) of the bioluminescent signal will be observed. Control strain can thus be used to correct the signal emitted by the biosensor.

In addition, also commercially available mammalian or human cell lines specific for polyhalogenated hydrocarbons can be used (Scippo M L, Eppe G, De Pauw E, Maghuin-Rogister G. DR-CALUX((R)) screening of food samples: evaluation of the quantitative approach to measure dioxin, furans and dioxin-like PCBs. Talanta. 2004 Aug. 8; 63(5):1193-202).

In an exemplary, simplified form of the present invention, as illustrated in FIG. 1, the portable device comprises a platform of cells (e.g., eight different cell lines that allow the analysis of 8 different analytes in the same sample) engineered to express luciferase as reporter gene in the presence of target analytes thus allowing a multiparametric analysis.

Typically, the device comprises a disposable plastic cartridge with genetically engineered cells (e.g., bacterial or yeast cells) immobilized in the matrix object of the present invention, that maintains cell vitality, an holder for the cartridge containing the light detectors (e.g., an array of avalanche diodes) in close contact with the support where cells are immobilized, a microfluidic system with pumps for the introduction of the sample and substrate for bioluminescence emission and a software for data elaboration.

Cells immobilized into the device are engineered with standard molecular biology procedures that depend on the type of target analyte and cell (e.g., bacteria, yeast).

The following examples illustrate the invention more in detail.

### Example 1

A device as illustrated in FIG. 1 was developed.

The device comprises a plastic cartridge with a matrix of 10×8 wells: each recombinant cell line is immobilized in a separate well, an array of ultrasensitive light detectors (e.g., avalanche photodiodes), a microfluidic system with pumps for the introduction of the sample, reference standard solutions, and substrate for bioluminescence emission (e.g., D-luciferin, if firefly luciferase is used as reporter protein). This configuration allows to use up to 10 different recombinant cell lines.

As shown in FIG. 1A, immobilized engineered cells are contained in the disposable transparent plastic-made cartridge, in which different cell strains are immobilized within separate microwells and a microfluidic system allows the introduction of the sample, reference standard solutions (with high and low concentration of target analytes), and substrate for bioluminescence emission.

Each solution is added in duplicate through a system of pumps or by manual dispensing. Total volume of the fluids in the channels varies from 100 μL to 1 mL. FIG. 1.B. shows a lateral view of the device. The dimension of the disposable cell cartridge are approximately 50×40 mm with microwells for cell immobilization of a diameter of about 3-4 mm with a total volume of approximately 50-70 μL. In the example there is a 10×8 matrix of wells: each recombinant cell line will be immobilized in 8 wells so that each sample can be assayed in duplicate: 2 wells for high concentration analyte reference standard, 2 wells for low concentration reference standard, 4 wells for the analysis of two samples in duplicate.

Using this geometry a total of 10 different recombinant cell lines can be used.

The device allows analyzing both liquid samples and solid ones such as sediments or other complex matrices previously treated to release the analyte in solution.

For example, the procedure for analysis of heavy metals in sediments is based on a pre-treatment of samples before the analysis: 1 g of lyophilized sediment is added to 9 mL of bidistilled water and kept with stirring at +25° C. for 24 hrs to release heavy metal in solution in a hydrosoluble form. Then serial dilutions (1:2, 1:10, 1:50) are prepared and incubated with immobilized cells for a period ranging from 30 min to 3 hrs at room temperature)(20-30°. Incubation times vary according to incubation temperature and the type of immobilized cells. For example a 2 hrs incubation is sufficient to obtain good detection limits with bacterial and yeast biosensors.

After incubation, the properly designed fluidic system, made with commercially available components, allows substrate addition (e.g., D-luciferin in citrate buffer at pH 5.0) to all channels. The use of this buffer allows the entrance of D-luciferin into cells thus avoiding the need for cell lysis before the measurements.

An array of diodes (commercially available or customized) allows detection of bioluminescent signal produced by single wells containing cells. A software provides data elaboration with the calculation of a correction factor (CF) for each standard solution or sample to take into account changes in cell vitality (e.g., inhibitory effects caused by toxic substances), by applying the following formula:

$$C F = \frac{{L}_{B}}{{L}_{S}}$$

where L_{B} is the luminescence of the control strain (e.g., constitutively expressing luciferase) with blank solvent and L_{S} is the luminescence of the control strain with the sample.

The bioluminescent signal obtained with the biosensors (SL_{S}) for each standard solution or sample is then multiplied by its CF to obtain the corrected light signal (CLS_{S}) which is:

LCS_{S}=CF×LS_{S}

For each calibration curve a limit of detection is calculated (LOD) according to the standard deviation on the blank signal measured with the calibration curve. It is provided by the analyte concentration corresponding to the signal X_{LOD}:

$${X}_{LOD} = 2 \frac{{X}_{B} + 3 SD}{{X}_{B}}$$

with X_{B}=mean of blank signals measured with the bacterial biosensor and SD=blank standard deviation.

Then, CLS is normalized, by setting the blank signal equal to 1. In particular, the normalized luminescence (NL) is calculated as follows:

$$LN = \frac{S L {C}_{S}}{{SL}_{B}}$$

where SL_{B} is the luminescent signal obtained measuring the blank with the bacterial biosensor.

The calibration curve of the biosensor is then reported in a graph with NL values in function of the decimal logarithm of the final concentration (dilution 1:100 v/v of the standard) of the analyte.

Cell vitality was evaluated for more than 30 days and no significant decreases were detected in bioluminescent emission, demonstrating that the matrix object of the present invention can effectively maintain cell vitality.

FIG. 2 shows bioluminescent signal of bacteria constitutively expressing luciferase immobilized with the matrix object of the present invention and kept at 4° C. for more than 1 month.

### Example 2

A calibration curve for copper was performed by employing recombinant E. coli cells specific for Cu²⁺ and a control strain that constitutively express firefly luciferase. The use of a control strain allows to correct the analytical signal with a detection limit of 0.05 ppm for Cu²⁺.

Cells are grown in selective liquid medium at 37° C. (e.g., Luria Bertani Broth with antibiotic for plasmid maintenance) for each strain up to an optical density at 600 nm in the range 0.6-0.8.

Then, a buffered solution at pH 6.5-7.5 is added to each cell suspension containing nutrients and is mixed 1:2-1:4 with the immobilization matrix.

A solution containing 1% atelocollagen in PBS (NaCl 137 mM, KCl 2.7 mM, NaH₂PO₄ 1.4 mM, Na₂HPO₄ 4.3 mM) at pH 7.4 with decorin final concentration 15% w/w atelocollagen.

Polyvinylpyrrolidone (PVP) and modified polysiloxanes (e.g., polydimethylsiloxane, tetraethyl-orthosilicate) are added to a final concentration of 1% and 0.4%, respectively.

### Example 3

Analogously to Example 2, a matrix with the biosensor specific for mercury was prepared.

As shown in FIG. 3, calibration curves for Hg²⁺ were obtained with non-immobilized bacteria (in selective ampicillin LB Broth liquid culture (—♦—), with bacteria immobilized in the matrix object of the present invention (—▴—) and bacteria immobilized in conventional matrix (LB/agar —▪—).

### Example 4

Analogously to Example 3, but with slight modifications to single components of the immobilization matrix (2% atelocollagen and 0.5% polydimethylsiloxane), a biosensor based on yeast cells specific to estrogens was immobilized.

Recombinant cells are grown in SC liquid culture (synthetic complete, selective for plasmid auxotrophies) at 30° C. up to an optical density at 600 nm of 0.6. Then, cells are immobilized as previously described. Calibration curves for 17-β estradiol showed a limit of detection similar to that obtained with liquid cultures (0.01 nM).

### Example 5

Precision of the method was evaluated both intra-assay and inter-assay (Table 1) for three biosensors, based on the use of three different bacterial strains: arsenite biosensor (E. coli), cadmium biosensor (B. subtilis) and lead biosensor (E. coli). In particular, the response of the bacterial biosensor was evaluated in four replicates of metal solution at different concentrations (low, medium and high) in the same experiment and in four different series of experiments in which different cell cultures were used. Reproducibility was good, with variation coefficients always less than 15% and 30% in the same experiment and in different experiments, respectively. These data confirmed that the immobilization procedure is sufficiently stable and reproducible from an analytical point of view.

TABLE 1

| | Metal concentration | | Intra-assay CV % | Inter-assay CV % |
|---|---|---|---|---|
| E. coli | AsO₂⁻ 7 × 10⁻⁵ | ppm | 12.2 | 27.7 |
| for AsO₂⁻ | AsO₂⁻ 7 × 10⁻⁴ | ppm | 9.3 | 24.5 |
| | AsO₂⁻ 7 × 10⁻³ | ppm | 11.9 | 21.9 |
| B. subtilis | Cd²⁺ 1.1 × 10⁻⁵ | ppm | 12.3 | 18.6 |
| for Cd²⁺ | Cd²⁺ 1.1 × 10⁻⁴ | ppm | 9.4 | 23.9 |
| | Cd²⁺ 1.1 × 10⁻³ | ppm | 8.7 | 25.8 |
| E. coli | Pb²⁺ 6 × 10⁻² | ppm | 9.6 | 17.6 |
| for Pb²⁺ | Pb²⁺ 2 × 10⁻¹ | ppm | 8.2 | 19.9 |
| | Pb²⁺ 2 | ppm | 10.1 | 22.3 |

Evaluation of repeatability (intra-assay) and reproducibility (inter-assay) of calibration curves determined on the base of low, medium and high concentrations of metal.

### Example 6

Analogously to Example 4, a yeast biosensor specific for androgens was immobilized with the matrix object of the present invention. Recombinant cells were grown in liquid SC medium (synthetic complete, selective for plasmid auxotrophies) at 30° C. up to an optical density at 600 nm of 0.6. Then, cells were immobilized as previously described.

FIG. 4 shows a typical calibration curve for testosterone obtained with the yeast strain responding to androgens immobilized in the matrix object of the invention. Cells were immobilized and kept at 4° C. for 2 weeks, then incubated with testosterone serial dilution for 2 hrs and light emission was measured immediately after addition of D-luciferin substrate with a CCD camera.

### INDUSTRIAL APPLICATIONS

The present invention finds applications in all those fields in which the detection of toxic substances or of compounds with specific biological activity is required. In particular, the invention applies to:
Screening of environmental samples: influents and effluents of water treatment plants, sea and river waters, sediments.
Screening of clinical samples: serum; blood, urine, saliva. (e.g., for anti-doping analysis)
Screening of food samples (baby food, mineral waters in PET and other plastic packages to monitor release of bisphenol A and other plasticizers with pseudo-hormonal activities)
Pre-screening of new drugs and pharmacologically active molecules (e.g., for detecting hormonal activity of new drugs for substitutive hormonal therapies)

1. Portable device for the detection of analytes comprising at least a whole-cell biosensor, characterized in that said cells are immobilized on a transparent, inert matrix which allows maintenance of cell viability, said matrix comprising:
a) a natural, polymeric component, said natural component is a mixture of collagen and/or enzyme degradation derivatives thereof and a proteoglycan, and optionally a vegetal mucilage, and
b) a synthetic, polymeric component, said synthetic component is a mixture of a vinyl polymer and a polysiloxane, optionally modified and/or crosslinked with an orthosilicate.

2. Device according to claim 1, wherein said natural component a) is a mixture of atelocollagen and decorin and said synthetic component b) is a mixture of polyvinylpyrrolidone and dimethylsiloxane and tetraethylorthosilicate.

3. Device according to claim 1, wherein said biosensor is a multiplicity of biosensors for a plurality of analytes.

4. Device according to claim 3, wherein said multiplicity of biosensors allows multiparametric, simultaneous detection of said analytes.

5. Device according to claim 1, wherein said biosensor is a bacteria cell or a yeast cell.

6. Device according to claim 1, wherein said biosensor is bioluminescent.

7. Device according to claim 6, wherein said biosensor is spontaneously bioluminescent.

8. Device according to claim 6, wherein said bioluminescent biosensor is a cell engineered with a gene reporter, which expresses luciferase in the presence of at least one of said analytes.

9. Device according to claim 1, wherein said analytes are toxic substances.

10. Device according to claim 6, wherein said biosensor is selected from the group consisting of: bioluminescent bacteria specific for heavy metals, bioluminescent yeasts specific for endocrine disruptors, xenobiotics and genotoxic compounds.

11. Matrix comprising:
a) a natural, polymeric component, said natural component is a mixture of collagen and/or enzyme degradation derivatives thereof and a proteoglycan, and optionally a vegetal mucilage, and
b) a synthetic, polymeric component, said synthetic component is a mixture of a vinyl polymer and a polysiloxane, optionally modified and/or crosslinked with an orthosilicate.
